(19) United States
(12) Patent Application Publication
(10) Pub. No.: US 2010/0010263 A1
(43) Pub. Date: Jan. 14, 2010

(54) Process For Preparing Substituted Phenylhydrazines

(75) Inventors: Thomas Zierke, Bohl-lggelheim (DE); Michael Rack, Eppelheim (DE)

Correspondence Address: BRINKS, HOFER, GILSON & LIONE, P.O. BOX 1340, MORRISVILLE, NC, 27560, US

(73) Assignee: BASF SE, Ludwigshafen (DE)

(21) Appl. No.: 12/529,066

(22) PCT Filed: Feb. 27, 2008
(86) PCT No.: PCT/EP2008/052346

§ 371 Date: Aug. 28, 2009

(30) Foreign Application Priority Data
Mar. 16, 2007 (EP) 07104341.8

(51) Int. Cl. C07C 241/02

(52) U.S. Cl. 564/314

(57) ABSTRACT

This invention relates to a process for preparing substituted phenylhydrazines of the formula I wherein R has the meaning as indicated in the description, comprising reacting a dichlorofluorobenzene of the formula II with a hydrazine source selected from hydrazine, hydrazine hydrate and acid addition salts of hydrazine and optionally being carried out in the presence of at least one organic solvent.

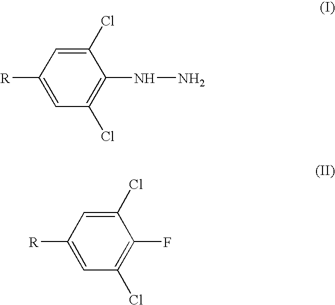

The present invention relates to a process for preparing substituted phenylhydrazines of the formula I

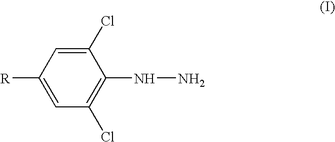

wherein R has the meaning as given below.

The substituted phenylhydrazines of the formula I are important intermediate products for the preparation of various pesticides (see, for example, WO 00/59862, EP-A 0 187 285, WO 00/46210, EP-A 096645, EP-A 0954144 and EP-A 0952145).

EP-A 0 224 831 describes the preparation of various phenylhydrazines by reacting halogenated aromatic compounds with hydrazine or hydrazine hydrate. According to preparation example V-1,2,6-dichloro-3-fluoro-4-trifluoromethyl phenylhydrazine can be prepared by reacting 3,5-dichloro-2,4-difluorobenzotrifluoride with hydrazine hydrate in ethanol under reflux conditions.

Methods for preparing the substituted phenylhydrazines of the formula I are also known from the prior art.

For example, EP-A 0 187 285 describes the preparation of 2,6-dichloro-4-(trifluoromethyl)phenylhydrazine (synonym name: 1-[2,6-dichloro-4-(trifluoromethyl) phenyl]hydrazine) by the reaction of 3,4,5-trichlorotrifluoromethyl-benzene (herein also referred to as 3,4,5-trichlorobenzotrifluoride) with 5 molar equivalents of hydrazine hydrate in pyridine at a temperature of from 115 to 120° C. for 48 hours. The desired end product is obtained in a yield of 83% with a purity of 90% as determined by gas chromatography (see preparation example 1).

However, the process described in EP-A 0 187 285 requires relatively high temperatures and relatively long reaction times. Another disadvantage of this process is the limited selectivity for the desired end product. Furthermore, the hydrazine source must be used in a relatively high excess amount. However, the excess of hydrazine subsequently has to be worked up or destroyed, which is costly in an economic sense and unfavorable from a viewpoint of environmental protection. In addition, the above process is conducted in pyridine as solvent, the recovery and removal of which is also problematic on an industrial scale.

It is therefore an object of the present invention to provide an improved process for preparing the substituted phenylhydrazines of the formula I, in particular to find procedures which can be performed at moderate temperatures and in shorter reaction times, while simultaneously achieving an economically acceptable yield and a higher selectivity of the desired end product. It is another object of this invention to reduce the environmental impact of the preparation of the substituted phenylhydrazines of the formula I.

These and further objects can be achieved in whole or in part by a process for preparing substituted phenylhydrazines of the formula I

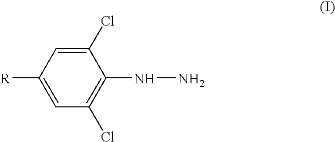

wherein R is C₁-C₄ haloalkyl, C₁-C₄ haloalkoxy or C₁-C₄ haloalkylthio, said process comprising reacting a dichlorofluorobenzene of the formula II

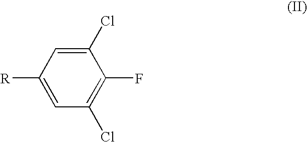

wherein R has the same meaning as defined above, with a hydrazine source selected from hydrazine, hydrazine hydrate and acid addition salts of hydrazine and optionally being carried out in the presence of at least one organic solvent.

It has surprisingly been found that, by using the dichlorofluorobenzene of the formula II as starting material, the substituted phenylhydrazines of the formula I can be obtained under milder conditions and with a higher conversion and selectivity when compared to the prior art processes. In addition, the reaction can be carried out in a wide variety of organic solvents ranging from non-polar solvents to highly polar solvents. This broadens the choice of organic solvents that can be employed for the synthesis of the substituted phenylhydrazines of the formula I, so as to avoid the use of environmentally unfavorable or expensive solvents, such as pyridine. Furthermore, the amount of the hydrazine source to be reacted with the starting material can be significantly reduced so as to improve recovery and waste disposal and to minimize costs.

The term “C₁-C₄ haloalkyl” as used herein refers to a C₁-C₄ alkyl group (as defined hereinbelow) which additionally contains one or more, e.g. 2, 3, 4, 5, 6 or 7 halogen atom(s) (as defined hereinbelow), e.g. mono- di- and trifluoromethyl, mono-, di- and trichloromethyl, 1-fluoroethyl, 1-chloroethyl, 2-fluoroethyl, 2-chloroethyl, 1,1-difluoroethyl, 1,1-dichloroethyl, 1,2-difluoroethyl, 1,2-dichloroethyl, 2,2-difluoroethyl, 2,2-dichloroethyl, 2,2,2-trifluoroethyl, 2,2,2-trichloroethyl and heptafluoroisopropyl.

The term “C₁-C₄ alkyl”, as used herein in the related term “C₁-C₄ haloalkyl”, refers to straight or branched aliphatic alkyl groups having from 1 to 4 carbon atoms, e.g. methyl, ethyl, propyl, isopropyl, n-butyl, sec-butyl and tert-butyl.

The term “halogen” is taken to mean fluorine, chlorine, bromine, and iodine.

The term “C₁-C₄ haloalkoxy” as used herein refers to a C₁-C₄ alkoxy group (as defined hereinbelow), which additionally contains one or more, e.g. 2, 3, 4, 5, 6 or 7 halogen atom(s), as defined above, e.g. mono- di- and trifluoromethoxy, mono- di- and trichloromethoxy, 1-fluoroethoxy, 1-chloroethoxy, 2-fluoroethoxy, 2-chloroethoxy, 1,1-difluoroethoxy, 1,1-dichloroethoxy, 1,2-difluoroethoxy, 1,2-dichloroethoxy, 2,2-difluoroethoxy, 2,2-dichloroethoxy, 2,2,2-trifluoroethoxy, 1,1,2,2-tetrafluoroethoxy, 2,2,2-trichloroethoxy, 1,1,1,2,3,3-hexafluoroisopropoxy, 1,1,2,3,3,3-hexafluoroisopropoxy, 2-chloro-1,1,2-trifluoroethoxy and heptafluoroisopropoxy.

The term “C₁-C₄ haloalkylthio” as used herein refers to a C₁-C₄ alkylthio group (as defined hereinbelow), which additionally contains one or more, e.g. 2, 3, 4, 5, 6 or 7 halogen atom(s), as defined above, e.g. mono- di- and trifluoromethylthio, mono- di- and trichloromethylthio, 1-fluoroethylthio, 1-chloroethylthio, 2-fluoroethylthio, 2-chloroethylthio, 1,1-difluoroethylthio, 1,1-dichloroethylthio, 1,2-difluoroethylthio, 1,2-dichloroethylthio, 2,2-difluoroethylthio, 2,2-dichloroethylthio, 2,2,2-trifluoroethylthio, 1,1,2,2-tetrafluoroethylthio, 2,2,2-trichloroethylthio, 1,1,1,2,3,3-hexafluoroisopropylthio, 1,1,2,3,3,3-hexafluoroisopropylthio, 2-chloro-1,1,2-trifluoroethylthio and heptafluoroisopropylthio.

The term “C₁-C₄ alkoxy”, as used herein in the related term “C₁-C₄ haloalkoxy”, refers to a C₁-C₄ alkyl group (as defined above) which is linked via an oxygen atom, e.g. methoxy, ethoxy, propoxy, isopropoxy, n-butoxy, sec-butoxy, iso-butoxy and tert-butoxy.

The term “C₁-C₄ alkylthio”, as used herein in the related term “C₁-C₄ haloalkylthio”, refers to a C₁-C₄ alkyl group (as defined above) which is linked via a sulphur atom, e.g. methylthio, ethylthio, propylthio, isopropylthio, n-butylthio, sec-butylthio, iso-butylthio and tert-butylthio.

For the process according to the invention, it has been found to be particularly advantageous when R in formula I and accordingly also in formula II is C₁-C₄-haloalkyl, in particular trifluoromethyl.

A particularly preferred embodiment of the present invention, therefore, provides a process for preparing 2,6-dichloro-4-(trifluoromethyl)phenylhydrazine of the formula I-1

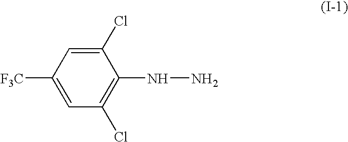

said process comprising reacting 1,3-dichloro-2-fluoro-5-trifluoromethylbenzene of the formula II-1 (hereinafter also referred to as “3,5-dichloro-4-fluorobenzotrifluoride”)

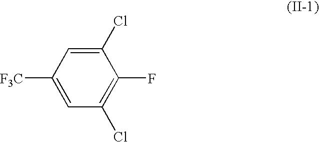

with a hydrazine source as defined herein and optionally being carried out in the presence of at least one organic solvent.

The dichlorofluorobenzenes of the formula II (such as, e.g., 1,3-dichloro-2-fluoro-5-trifluoromethylbenzene of the formula II-1) are known compounds and may be prepared by known methods, such as those described in EP-A 0 034 402, U.S. Pat. Nos. 4,388,472, 4,590,315 and Journal of Fluorine Chemistry, 30 (1985), pp. 251-258, or in an analogous manner.

In general, the hydrazine source is used in an at least equimolar amount or in a slight excess, relative to the dichlorofluorobenzene of the formula II. Preference is given to using 1 to 6 moles, in particular from 1 to 4 moles, and more preferably from 1 to 3 moles of the hydrazine source, relative to 1 mole of the dichlorofluorobenzene of the formula II.

In a preferred embodiment, the dichlorofluorobenzene of the formula II (in particular 1,3-dichloro-2-fluoro-5-trifluoromethylbenzene of the formula II-1) is reacted with hydrazine hydrate. The amount of hydrazine hydrate is generally from 1 to 6 moles, in particular from 1 to 4 moles and more preferably from 1 to 3 moles, relative to 1 mole of the dichlorofluorobenzene of the formula II (in particular 1,3-dichloro-2-fluoro-5-trifluoromethylbenzene of the formula II-1).

The term “acid addition salts of hydrazine” refers to hydrazine salts formed from strong acids such as mineral acids (e.g. hydrazine sulfate and hydrazine hydrochloride).

The process according to the invention may in principle be carried out in bulk, but preferably in the presence of at least one organic solvent.

Suitable organic solvents are practically all inert organic solvents including cyclic or aliphatic ethers such as dimethoxyethan, diethoxyethan, bis(2-methoxyethyl) ether (diglyme), triethyleneglycoldimethyl ether (triglyme), dibutyl ether, methyl tert-butyl ether, tetrahydrofuran, 2-methyltetrahydrofuran, dioxane and the like; aromatic hydrocarbons such as toluene, xylenes (ortho-xylene, meta-xylene and para-xylene), ethylbenzene, mesitylene, chlorobenzene, dichlorobenzenes, anisole and the like; alcohols such as methanol, ethanol, n-propanol, isopropanol, n-butanol and the like; tertiary C₁-C₄ alkylamines such as triethylamine, tributylamine, diisoproylethylamine and the like; heterocyclic aromatic compounds such as pyridine, 2-methylpyridine, 3-methylpyridine, 5-ethyl-2-methylpyridine, 2,4,6-trimethylpyridine (collidine), lutidines (2,6-dimethylpyridine, 2,4-dimethylpyridine and 3,5-dimethylpyridine), 4-dimethylaminopyridine and the like; and any mixture of the aforementioned solvents.

Preferred organic solvents are cyclic ethers (in particular those as defined hereinabove), alcohols (in particular those as defined hereinabove), aromatic hydrocarbons (in particular those as defined hereinabove) and heterocyclic aromatic compounds (in particular those as defined hereinabove) and any mixture thereof. More preferably, the organic solvent is selected from cyclic ethers (in particular from those as defined hereinabove) and aromatic hydrocarbons (in particular from those as defined hereinabove), and any mixture thereof.

Thus, a broad variety of organic solvents can surprisingly be utilized for the preparation of the substituted phenylhydrazines of the formula I including non-polar solvents, weakly polar solvents, polar protic solvents and polar aprotic solvents.

In a preferred embodiment, non-polar or weakly polar organic solvents having a dielectric constant of not more than 12, preferably not more than 8 at a temperature of 25° C. are used in the process according to this invention. Such non-polar or weakly polar organic solvents can be selected from among a variety of organic solvents known to a skilled person, in particular from those listed hereinabove. Specific examples of organic solvents fulfilling the above requirements include aromatic hydrocarbons, in particular toluene (having a dielectric constant of 2.38 at 25° C.), and cyclic ethers, in particular tetrahydrofuran (having a dielectric constant of 7.58 at 25° C.).

Preferred organic solvents are aromatic hydrocarbons, in particular those as listed hereinabove and any mixture thereof. Toluene is most preferred among the aromatic hydrocarbons.

Preference is also given to heterocyclic aromatic compounds, in particular those as listed hereinabove and any mixture thereof, and most preferably pyridine.

The most preferred organic solvents are cyclic ethers, in particular cyclic ethers having from 4 to 8 carbon atoms, and more preferably tetrahydrofuran.

The organic solvent is generally used in an amount of 1 to 15 moles, in particular from 2 to 10 moles, and more preferably from 3 to 8 moles, relative to 1 mole of the dichlorofluorobenzene of the formula II.

The process according to the invention may be conducted at a temperature up to the boiling point of the reaction mixture. Advantageously, the process can be carried out at an unexpectedly low temperature, such as below 60° C. The preferred temperature range is from 0° C. to 60° C., more preferably 10° C. to 55° C., yet more preferably 15° C. to 50° C., even more preferably 15° C. to 45° C. and most preferably 20° C. to 40° C.

The reaction of the dichlorofluorobenzene of the formula II with the hydrazine source can be carried out under reduced pressure, normal pressure (i.e. atmospheric pressure) or increased pressure. Preference is given to carrying out the reaction in the region of atmospheric pressure.

The reaction time can be varied in a wide range and depends on a variety of factors, such as, for example, the reaction temperature, the organic solvent, the hydrazine source and the amount thereof. The reaction time required for the reaction is generally in the range from 1 to 120 hours, in particular 12 to 120 hours, and more preferably 24 to 120 hours.

The dichlorofluorobenzene of the formula II and the hydrazine source may be contacted together in any suitable manner. Frequently, it is advantageous that the dichlorofluorobenzene of the formula II is initially charged into a reaction vessel, optionally together with the organic solvent desired, and the hydrazine source is then added to the resulting mixture.

The reaction mixture can be worked up and the substituted phenylhydrazine of formula I can be isolated therefrom by using known methods, such as washing, extraction, precipitation, crystallization and distillation.

If desired, the substituted phenylhydrazine of formula I can be purified after its isolation by using techniques that are known in the art, for example by distillation, recrystallization and the like.

The conversion of the dichlorofluorobenzene of the formula II (in particular of 1,3-dichloro-2-fluoro-5-trifluoromethylbenzene of the formula II-1) in the process of this invention usually exceeds 10%, in particular 50%, more preferably 75% and even more preferably 90%.

The conversion is usually measured by evaluation of area-% of signals in the gas chromatography assay of a sample taken from the reaction solution (hereinafter also referred to as “GC area-%”). For the purposes of this invention, conversion is defined as the ratio of the GC area-% of the substituted phenylhydrazines of the formula I (in particular the GC area-% of 2,6-dichloro-4-(trifluoromethyl) phenylhydrazine of the formula I-1) against the sum of the GC area-% of the substituted phenylhydrazines of the formula I (in particular the GC area-% of 2,6-dichloro-4-(trifluoromethyl) phenylhydrazine of the formula I-1) and the GC area-% of not converted dichlorofluorobenzene of the formula II (in particular the GC area-% of not converted 1,3-dichloro-2-fluoro-5-trifluoromethylbenzene of the formula II-1), with said ratio being multiplied by 100 to obtain the percent conversion.

Combinations of preferred embodiments with other preferred embodiments are within the scope of the present invention.

The process according to the invention has a number of advantages over the procedures hitherto used for the preparation of the substituted phenylhydrazines of the formula I. Firstly, it has been shown that virtually complete conversion of the dichlorofluorobenzene of the formula II (in particular of 1,3-dichloro-2-fluoro-5-trifluoromethylbenzene) can be achieved even at relatively low temperatures (e.g. 20° C. to 30° C.) and shorter reaction times. Secondly, the process according to the invention results in a very high selectivity to the desired product of value. Thus, since no significant amounts of undesired isomers are formed, the reaction mixture can be used in subsequent reactions without cost-intensive work-up and purification measures. For example, if 1,3-dichloro-2-fluoro-5-trifluoromethylbenzene of the formula II-1 is reacted with the hydrazine source (especially with hydrazine hydrate), the selectivity to the desired 2,6-dichloro-4-(trifluoromethyl)phenylhydrazine of the formula I-1 is surprisingly high. No substituted phenylhydrazine resulting from the displacement of chlorine instead of the fluorine atom in 1,3-dichloro-2-fluoro-5-trifluoromethylbenzene is observed. The only by-product, which is observed in some cases in a very small amount, is the mono de-chlorinated analogue of the aimed product, i.e. 2-chloro-4-(trifluoromethyl) phenylhydrazine. Also, high conversions and selectivities are achievable in a wide variety of solvents. Furthermore, the use of cyclic ethers such as tetrahydrofuran and the use of a lower excess of the hydrazine source offer advantages compared to the prior art. This saves raw material costs and reduces also the efforts for waste disposal. In summary, the process of the present invention provides a more economic and industrially more feasible route to the substituted phenylhydrazines of formula I.

The following Examples are illustrative of the process of this invention, but are not intended to be limiting thereof. The invention is further illustrated by the following Comparative Examples (not of the invention).

### EXAMPLE 1

### Preparation of 2,6-dichloro-4-(trifluoromethyl) phenylhydrazine of the formula I-1 in tetrahydrofurane

2.5 g (11 mmole) of 1,3-dichloro-2-fluoro-5-trifluoromethylbenzene (98% purity) of the formula II-1 were dissolved in 5.3 g (74 mmole) of tetrahydrofuran. To this solution were added 2.1 g (41 mmole) of hydrazine hydrate (100%). The resulting mixture was stirred at 25° C. for 91 hours. Thereafter, an organic phase of 7.6 g was separated, which contained the product 2,6-dichloro-4-(trifluoromethyl) phenylhydrazine as a 33.5 wt-% solution in tetrahydrofuran, meaning that a yield of 99% was obtained. The solvent was stripped off. A sample of the solid residue was used for ¹H-NMR spectroscopy to demonstrate the identity of the product.

¹H-NMR (400 MHz, CDCl₃): δ/ppm =4.05 (s, 2H); 5.9 (s, 1H); 7.5 (s, 2H)

### EXAMPLE 2

### Preparation of 2,6-dichloro-4-(trifluoromethyl) phenylhydrazine of the formula I-1 in tetrahydrofurane (amount of hydrazine hydrate: 2.1 equivalents)

2.5 g (11 mmole) of 1,3-dichloro-2-fluoro-5-trifluoromethylbenzene (98% purity) of the formula II-1 were dissolved in 5.3 g (74 mmole) of tetrahydrofuran. To this solution were added 1.1 g (22 mmole) of hydrazine hydrate (100%). The resulting mixture was stirred at 25° C. for 24 h and at 50° C. for 2 h. Thereafter, an organic phase of 7.6 g was separated, which contained the product 2,6-dichloro-4-(trifluoromethyl) phenylhydrazine as a 29.5 wt-% solution in tetrahydrofuran, meaning that a yield of 87% was obtained.

### COMPARATIVE EXAMPLE 1

### Preparation of 2,6-dichloro-4-(trifluoromethyl) phenylhydrazine of the formula I-1 from 3,4,5-trichlorobenzotrifluoride in tetrahydrofurane

10 g (40 mmole) of 3,4,5-trichlorobenzotrifluoride (99.7% purity) were dissolved in 30 g (417 mmole) of tetrahydrofurane. To this solution were added 8 g (160 mmole) of hydrazine hydrate (100%). The resulting mixture was stirred at 50° C. for 24 hours. Thereafter, an organic phase of 40.7 g was separated. The solution obtained by this separation contained the product 2,6-dichloro-4-(trifluoromethyl)phenylhydrazine in an amount of 0.9 wt-% and the starting material 3,4,5-trichlorobenzotrifluoride in an amount of 27.1 wt-%, meaning that a product yield not higher than 3.7 % was obtained.

### EXAMPLE 3

### Preparation of 2,6-dichloro-4-(trifluoromethyl) phenylhydrazine of the formula I-1 in pyridine

5.0 g (21 mmole) of 1,3-dichloro-2-fluoro-5-trifluoromethylbenzene (98% purity) were dissolved in 11.7 g (147 mmole) of pyridine. To this solution were added 4.2 g (84 mmole) of hydrazine hydrate (100%). The resulting mixture was stirred at 25° C. for 20 hours. Gas chromatographic assay of a sample showed 97% conversion. After additional 73 hours at 25° C. and 5 hours at 50° C., an organic phase of 16.6 g was separated, which contained the product 2,6-dichloro-4-(trifluoromethyl)phenylhydrazine as a 29.4 wt-% solution in pyridine, meaning that a yield of 95% was obtained.

### EXAMPLE 4

### Preparation of 2,6-dichloro-4-(trifluoromethyl) phenylhydrazine of the formula I-1 in pyridine (amount of hydrazine hydrate: 4 equivalents, reaction time: 6 hours, reaction temperature: 25° C.)

10 g (42 mmole) of 1,3-dichloro-2-fluoro-5-trifluoromethylbenzene (99% purity) were dissolved in 23.5 g (297 mmole) of pyridine. To this solution were added 8.5 g (170 mmole) of hydrazine hydrate (100%). The resulting mixture was stirred at 25° C. for 6 hours. Thereafter, an organic phase of 36.3 g was separated, which contained the product 2,6-dichloro-4-(trifluoromethyl) phenylhydrazine as a 25 wt-% solution in pyridine, meaning that a yield of 87% was obtained.

### COMPARATIVE EXAMPLE 2

### Preparation of 2,6-dichloro-4-(trifluoromethyl)phenylhydrazine of the formula I-1 from 3,4,5-trichlorobenzotrifluoride in pyridine (amount of hydrazine hydrate: 4 equivalents, reaction time: 24 hours, reaction temperature: 25° C.)

10 g (40 mmole) of 3,4,5-trichlorobenzotrifluoride (99.7% purity) were dissolved in 30 g (380 mmole) of pyridine. To this solution were added 8 g (160 mmole) of hydrazine hydrate (100%). The resulting mixture was stirred at 25° C. for 24 hours. Thereafter, an organic phase of 41.6 g was separated (lower phase). The solution obtained by this separation contained the product 2,6-dichloro-4-(trifluoromethyl) phenylhydrazine in an amount of 0.5 wt-% and the starting material 3,4,5-trichlorobenzotrifluoride in an amount of 26.4 wt-%, meaning that a product yield not higher than 2.5% was obtained.

### EXAMPLE 5

### Preparation of 2,6-dichloro-4-(trifluoromethyl) phenylhydrazine of the formula I-1 in pyridine (amount of hydrazine hydrate: 2.1 equivalents)

10 g (42 mmole) of 1,3-dichloro-2-fluoro-5-trifluoromethylbenzene (99% purity) were dissolved in 23.5 g (297 mmole) of pyridine. To this solution were added 4.5 g (90 mmole) of hydrazine hydrate (100%). The resulting mixture was stirred at 25° C. for 6 hours and then at 50° C. for 2 hours. Thereafter, an organic phase of 24.8 g was separated, which contained the product 2,6-dichloro-4-(trifluoromethyl)phenylhydrazine as a 32 wt-% solution in pyridine, meaning that a yield of 76% was obtained.

### EXAMPLE 6

### Preparation of 2,6-dichloro-4-(trifluoromethyl) phenylhydrazine of the formula I-1 in toluene

2.5 g (11 mmole) of 1,3-dichloro-2-fluoro-5-trifluoromethylbenzene (98% purity) were dissolved in 6.8 g (74 mmole) of toluene. To this solution were added 2.1 g (41 mmole) of hydrazine hydrate (100%). The resulting mixture was refluxed at 110° C. for 24 hours. Gas chromatrographic assay of a sample showed 97% conversion. Thereafter, the reaction mixture was worked up by addition of 22 g of toluene and 10 g of water. An organic phase of 28.5 g was separated, which contained the product 2,6-dichloro-4-(trifluoromethyl) phenylhydrazine as a 8.4 wt-% solution in pyridine, meaning that a yield of 93% was obtained.

### COMPARATIVE EXAMPLE 3

### Preparation of 2,6-dichloro-4-(trifluoromethyl)phenylhydrazine of the formula I-1 from 3,4,5-trichlorobenzotrifluoride in toluene

10 g (40 mmole) of 3,4,5-trichlorobenzotrifluoride (99.7% purity) were dissolved in 30 g (326 mmole) of toluene. To this solution were added 8 g (160 mmole) of hydrazine hydrate (100%). The resulting mixture was stirred at reflux (approx. 110° C.) for 24 hours. Thereafter, an organic phase of 39.4 g was separated. The solution obtained by this separation contained the product 2,6-dichloro-4-(trifluoromethyl)phenylhydrazine in an amount of 0.9 wt-% and the starting material 3,4,5-trichlorobenzotrifluoride in an amount of 26.3 wt-%, meaning that a product yield not higher than 3.6% was obtained.

1-11. (canceled)

12. A process for preparing substituted phenylhydrazines of the formula I 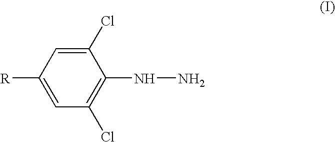
wherein R is C₁-C₄ haloalkyl, C₁-C₄ haloalkoxy or C₁-C₄ haloalkylthio,
said process comprising reacting a dichlorofluorobenzene of the formula II 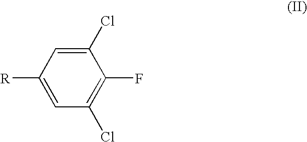
wherein R has the same meaning as defined above,
with a hydrazine source selected from hydrazine, hydrazine hydrate and acid addition salts of hydrazine and being carried out in the presence of at least one organic solvent.

13. The process of claim 12, wherein said organic solvent is selected from non-polar or weakly polar organic solvents having a dielectric constant of not more than 8 at a temperature of 25° C.

14. The process of claim 12, wherein said organic solvent is selected from cyclic ethers.

15. The process of claim 13, wherein said organic solvent is selected from cyclic ethers.

16. The process of claim 14, wherein said cyclic ether has 4 to 8 carbon atoms.

17. The process of claim 15, wherein said cyclic ether has 4 to 8 carbon atoms.

18. The process of claim 16, wherein said cyclic ether is tetrahydrofuran.

19. The process of claim 17, wherein said cyclic ether is tetrahydrofuran

20. The process of claim 12, wherein said reaction is carried out at a temperature in the range of from 15° C. to 45° C.

21. The process of claim 12, wherein said hydrazine source is hydrazine hydrate.

22. The process of claim 21, wherein said hydrazine hydrate is used in an amount of 1 to 6 moles, relative to 1 mole of the dichlorofluorobenzene of formula II.

23. The process of claim 21, wherein said hydrazine hydrate is used in an amount of 1 to 3 moles, relative to 1 mole of the dichlorofluorobenzene of formula II.

24. The process of claim 12, wherein R in the formulae I and II is C₁-C₄ haloalkyl.

25. The process of claim 24, wherein R in the formulae I and II is trifluoromethyl.
